SEQUENCE LISTING (13 sequences)

SEQ ID NO: 1 - protein, 20 aa, Artificial sequence
  Amino acids 82-101 of human caveolin-1
DGIWKASFTTFTVTKYWFYR

SEQ ID NO: 2 - protein, 44 aa, Artificial sequence
  Amino acids 135-178 of human caveolin-1
KSFLIEIQCTSRVYSIYVHTVCDPLFEAVGKIFSNVRINLQLEI

SEQ ID NO: 3 - protein, 20 aa, Artificial sequence
  Amino acids 55-74 of rat caveolin-3
DGVWRVSYTTFTVTKYWCYR

SEQ ID NO: 4 - protein, 22 aa, Artificial sequence
  Amino acids 108-129 of rat caveolin-3
KSYLIEIQCISHIYSLCIRTFC

SEQ ID NO: 5 - protein, 14 aa, Artificial sequence
  Amino acids 82-95 of human caveolin-1
DGIWKASFTTFTVT

SEQ ID NO: 6 - protein, 7 aa, Artificial sequence
  Amino acids 89-95 of human caveolin-1
FTTFTVT

SEQ ID NO: 7 - protein, 13 aa, Artificial sequence
  Amino acids 89-101 of human caveolin-1
FTTFTVTKYWFYR

SEQ ID NO: 8 - protein, 7 aa, Artificial sequence
  Chemically synthesized
ATTFTVT

SEQ ID NO: 9 - protein, 7 aa, Artificial sequence
  Chemically synthesized
FTTFAVT

SEQ ID NO: 10 - protein, 7 aa, Artificial sequence
  Chemically synthesized
FTTFTAT

SEQ ID NO: 11 - protein, 7 aa, Artificial sequence
  Chemically synthesized
FTTFTVA

SEQ ID NO: 12 - protein, 16 aa, Artificial sequence
  Homeodomain, internalization sequence
RQIKIWFQNRRMKWKK

SEQ ID NO: 13 - protein, 36 aa, Artificial sequence
  Antennapedia-caveolin-1 scaffolding domain fusion peptide
RQIKIWFQNRRMKWKKDGIWKASFTTFTVTKYWFYR

(19) United States
(12) Patent Application Publication
(10) Pub. No.: US 2015/0141340 A1
(43) Pub. Date: May 21, 2015

(54) METHOD OF TREATING AND PREVENTING OCULAR ANGIOGENESIS

(71) Applicant: Yale University, New Haven, CT (US)

(72) Inventor: William C. Sessa, Madison, CT (US)

(21) Appl. No.: 14/405,308

(22) PCT Filed: May 30, 2013
(86) PCT No.: PCT/US13/43307

§ 371 Date: Dec. 3, 2014

Related U.S. Application Data
(60) Provisional application No. 61/655,124, filed on Jun. 4, 2012.

(51) Int. Cl. C07K 14/47; A61K 38/17; C07K 7/08; C07K 7/06; A61K 38/10; A61K 45/06; A61K 38/08

(52) U.S. Cl. CPC C07K 14/47; A61K 45/06; A61K 38/1709; A61K 38/08; C07K 7/06; A61K 38/10; C07K 7/08
USPC 514/13.3; 514/20.8

(57) ABSTRACT

The present invention provides methods for inhibiting ocular angiogenesis, vascular leakage, and/or edema. The methods comprise administering to a subject an agent comprising a caveolin scaffolding domain. The present invention further encompasses methods of treating and/or preventing ophthalmic conditions that are associated with ocular angiogenesis, vascular leakage, or edema.

### CROSS-REFERENCE TO RELATED APPLICATION

This application is s a 35 U.S.C. §371 national phase application of, and claims priority to, PCT Application No. PCT/US2013/043307, filed May 30, 2013, which claims priority to U.S. Provisional Application Ser. No. 61/655,124, filed Jun. 4, 2012, the content of which is incorporated by reference herein in its entirety.

### STATEMENT REGARDING FEDERALLY SPONSORED RESEARCH OR DEVELOPMENT

This invention was made with government support under HL064793, HL061371, HL081190 and HL096670 awarded by National Institute of Health. The government has certain rights in the invention.

### BACKGROUND OF THE INVENTION

Ocular angiogenesis refers to the growth of blood vessels within the eye. While angiogenesis is a normal bodily function, abnormal angiogenesis can significantly contribute to a variety of disease states. Abnormal ocular angiogenesis is associated with a myriad of ophthalmic disorders and conditions, including macular degeneration. Damage to the existing blood vessels and the inappropriate development of new blood vessels are the hallmarks of various forms of blindness. Blood vessel development can occur in the retina, which occurs in a subset of ophthalmic conditions including diabetic retinopathy, which affects about 150 million patients (Frank, 2004, N Engl J Med., 350(1): 485-58). In age-related macular degeneration (AMD), neovascularization, defined as the development of new blood vessels in previously avascular tissue, occurs in the choroid layer adjacent to the retina (Jager et al., 2008, N Engl J Med., 358(24): 2606-2617). Excessive growth of blood vessels in the eye can result in leaking of blood and fluid, scar formation, and loss of vision. Ocular angiogenesis can cause irreversible visual impairment through the opacification of the cornea or alterations in the neuronal architecture of the retina (Qazi et al., 2009, J. Genet. 88: 495-515).

Current interventions or therapies for ocular angiogenesis include laser treatments and molecular therapies directed against vascular endothelial growth factor (VEGF). Laser surgery can provide some attenuation of neovascularization, but can result in harmful side effects including cataracts, hemorrhage, and retinal detachment (Alvarez et al., 2009, PLoS One, e7867). VEGF based therapies have included a variety of different strategies including siRNA, antibodies, and small molecule inhibitors. However, there are concerns regarding a strategy that is based upon antagonizing VEGF alone, as other pro-angiogenic factors are observed to be upregulated thereby decreasing the therapeutic efficacy of VEGF based therapies (Alvarez et al. 2009, PLoS One, e7867).

Thus, there is a need in the art to develop additional, superior methods to treat and prevent ocular angiogenesis. The present invention satisfies this unmet need.

### SUMMARY OF THE INVENTION

The invention provides a method of inhibiting ocular angiogenesis in a subject. In one embodiment, the method comprises administering an effective amount of an agent comprising a caveolin scaffolding domain to a subject in need thereof.

In one embodiment, the caveolin scaffolding domain is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11.

In one embodiment, the agent is a fusion peptide comprising at least one membrane translocation domain and at least one caveolin scaffolding domain selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11.

In one embodiment, the agent is administered via intraocular injection.

In one embodiment, the agent is administered in combination with an anti-angiogenic agent.

In one embodiment, the anti-angiogenic agent reduces the activity of vascular endothelial growth factor (VEGF).

The invention also provides a method of treating an ophthalmic condition in a subject. In one embodiment, the method comprises administering an effective amount of an agent comprising a caveolin scaffolding domain to a subject in need thereof, wherein the ophthalmic condition has at least one component selected from the group consisting of ocular angiogenesis, vascular leakage, and edema.

In one embodiment, the ophthalmic condition is selected from the group consisting of macular degeneration, diabetic retinopathy, diabetic macular edema, diabetic retina ischemia, diabetic retina oedema, proliferative diabetic retinopathy, birdshot disease, multifocal choroiditis, corneal graft rejection, corneal neovascularization, retinopathy of premature infants, retinal vein occlusion, neovascular glaucoma and sickle cell retinopathy, ischemic retinopathy, macular edema, intraocular neovascularization, choroidal neovascularization, pseudoxanthoma elasticum, optic disc drusen, traumatic eye injury.

The invention also provides a method of preventing an ophthalmic condition in a subject. In one embodiment, the method comprises administering an effective amount of an agent comprising a caveolin scaffolding domain to a subject in need thereof, wherein the ophthalmic condition has at least one component selected from the group consisting of ocular angiogenesis, vascular leakage, and edema.

The invention also provides a method of reducing vascular leakage and edema in a subject. In one embodiment, the method comprises administering an effective amount of an agent comprising a caveolin scaffolding domain to a subject in need thereof.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 comprises a graph depicting the results of experiments that demonstrate that cavtratin treatment reduces CNV induced by laser damage.

FIG. 2 comprises a graph depicting the results of experiments that demonstrate that cavtratin treatment reduces permeability induced after laser damage.

FIG. 3 comprises a graph depicting the results of experiments that demonstrate that cavtratin dose dependently reduces CNV to a level comparable to anti-VEGF.

FIG. 4 comprises a set of images that demonstrate that cavtratin dose dependently reduces CNV to a level comparable to anti-VEGF.

FIG. 5A and FIG. 5B depict the results of experiments which demonstrate that cavtratin synergizes with anti-VEGF. FIG. 5A comprises a graph that demonstrates that the combination of 0.05 nmol cavtratin and 5 μg anti-VEGF reduces CNV area more than control or either 0.05 nmol cavtratin or 5 μg anti-VEGF alone. FIG. 5B comprise a set of images of immunofluorescent labeling with isolectin b4, which demonstrate that cavtratin synergizes with anti-VEGF.

FIG. 6A and FIG. 6B depict the results of experiments which demonstrate that combination of cavtratin with anti-VEGF reduces CNV post damage. FIG. 6A comprises a graph that demonstrates that the combination of 5 nmol cavtratin and 10 μg anti-VEGF administered post damage reduces CNV area. FIG. 6B comprise a set of images of immunofluorescent labeling with isolectin b4, which demonstrate that the combination of cavtratin with anti-VEGF reduces CNV post damage.

FIG. 7 comprises a graph depicting the results of experiments which demonstrates that cavtratin treatment reduces neovascularization in a model of retinopathy of prematurity.

FIG. 8 comprise a set of images depicting the attenuation of extensive ischemia triggered neovascularization. The left panels are representative of two AP injected eyes and right panels are from cavtratin injections.

### DETAILED DESCRIPTION

The invention relates to methods of inhibiting ocular angiogenesis in a subject. In one embodiment, the methods comprise administering a composition comprising a caveolin scaffolding domain to a subject in need thereof. In one embodiment, the methods comprise administering a composition comprising a caveolin scaffolding domain and a membrane translocation domain to a subject in need thereof.

The invention is based on the discovery that a fusion peptide comprising an antennapedia homeodomain fused to acaveolin-1 scaffolding domain inhibits and prevents ocular angiogenesis. In some instances the fusion peptide is referred to as “AP-Cay.” Further, in some instances the fusion peptide is referred to as “Cavtratin.” Ocular angiogenesis contributes to a variety of eye disorders, and thus the present invention includes methods of treating eye disorders associated with ocular angiogenesis. For example, the present invention includes methods of treating macular degeneration, including age-related macular degeneration (AMD), dry AMD, and wet AMD.

In one embodiment, the invention provides compositions and methods for inhibiting angiogenesis (e.g., ocular angiogenesis choroidal neovasculariztion) in a subject in need thereof comprising administering to the subject a composition comprising a caveolin scaffolding domain sequence.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “amino acid sequence variant” refers to polypeptides having amino acid sequences that differ to some extent from a native sequence polypeptide. Ordinarily, amino acid sequence variants will possess at least about 70% homology, or at least about 80%, or at least about 90% homology to the native polypeptide. The amino acid sequence variants possess substitutions, deletions, and/or insertions at certain positions within the amino acid sequence of the native amino acid sequence.

As used herein, the term “binding” refers to the adherence of molecules to one another, such as, but not limited to, enzymes to substrates, antibodies to antigens, DNA strands to their complementary strands. Binding occurs because the shape and chemical nature of parts of the molecule surfaces are complementary. A common metaphor is the “lock-and-key” used to describe how enzymes fit around their substrate. The binding of the caveolin protein may occur at one or more domains of eNOS, such as, but not limited to, the oxygenase domain of eNOS and/or the reductase domain of eNOS.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

As used herein, the term “fusion peptide” or “fusion polypeptide” or “fusion protein” or “fusion peptidomimetic” or “fusion non-peptide-analog” refers to a heterologous peptide, heterologous polypeptide, heterologous protein, peptidomimetic, or non-peptide analog linked to a membrane translocation domain.

As used herein, the terms “conservative variation” or “conservative substitution” as used herein refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine, and the like.

As used herein, the term “caveolin scaffolding domain” refers to domains inclusive of putative scaffolding domains of any caveolin protein. Thus, the term as it used herein is not limited to putative scaffolding domains. Examples of caveolin scaffolding domains include, but are not limited to, the following:
a) Amino acids 82-101 of human caveolin-1 (⁸²DGIWKASFTTFTVTKYWFYR¹⁰¹) (SEQ ID NO: 1) or canine equivalents. The complete sequence of human Cav-1 can be found at GenBank Accession No. Z18951.
b) Amino acids 135-178 of human caveolin-1 (¹³⁵KSFLIEIQCTSRVYSIYVHTVCDPLFEAVGKIFSNVRINLQLEI¹⁷⁸) (SEQ ID. NO: 2) or canine equivalents.
c) Amino acids 55-74 of rat caveolin-3 (⁶⁵DGVWRVSYTTFTVTKYWCYR⁸⁴) (SEQ ID NO: 3) or human equivalents. The complete sequence of human Cav-3 can be found at GenBank Accession No. AF036366.1; 39-152, 32-373. The complete protein code for human Cav-3 can be found at GenBank Accession No. AAC39758.1.
d) Amino acids 108-129 of rat caveolin-3 (¹⁰⁹KSYLIEIQCISHIYSLCIRTFC₁₃₀) (SEQ ID NO: 4) or human equivalents.

In one embodiment, Caveolin scaffolding domains also include regions within SEQ ID NOs 1-4. For example, caveolin scaffolding domains also include distinct regions of SEQ ID NO: 1 including:
a) Amino acids 82-95 of human caveolin-1 (⁸²DGIWKASFTTFTVT⁹⁵) (SEQ ID NO: 5) or canine equivalents;
b) Amino acids 89-95 of human caveolin-1 (⁸⁹FTTFTVT⁹⁵) (SEQ ID NO: 6) or canine equivalents; and
c) Amino acids 89-101 of human caveolin-1 (⁸⁹FTTFTVTKYWFYR¹⁰¹) (SEQ ID NO: 7) or canine equivalents.

In another embodiment, Caveolin scaffolding domains also include sequences having one or more mutations. For example, caveolin scaffolding domains also include sequences with at least one point mutation in SEQ ID NO: 6 including:

a)⁸⁹ATTFTVT⁹⁵ (SEQ ID NO: 8) or canine equivalents;

b)⁸⁹FTTFAVT⁹⁵ (SEQ ID NO: 9) or canine equivalents;

c)⁸⁹FTTFTAT⁹⁵ (SEQ ID NO: 10) or canine equivalents; and

d)⁸⁹FTTFTVA⁹⁵ (SEQ ID NO: 11) or canine equivalents.

As used herein, the term “domain” refers to a part of a molecule or structure that shares common physicochemical features, such as, but not limited to, hydrophobic, polar, globular and helical domains or properties. Specific examples of binding domains include, but are not limited to, DNA binding domains and ATP binding domains.

As used herein, the term “heterologous peptide” refers to any peptide, polypeptide or protein whose sequence is chosen in such a way that the product of the fusion of this sequence with the membrane translocation domain has a sequence different from the wild-type sequence flanking any membrane translocation domain.

As used herein, the term “membrane translocation domain” refers to a peptide capable of permeating the membrane of a cell and which is used to transport attached peptides into a cell in vivo. Membrane translocation domains include, but are not limited to, the third helix of the antennapedia homeodomain protein (Derossi et al., (1994) J. Biol. Chem. 269, 10444-10450; U.S. Pat. Nos. 5,888,762 & 6,015,787); Tat derived peptides (Fawell et al. (1994) Proc. Natl. Acad. Sci. 91, 664-668); alpha helical amphipathic peptides (Oehlke et al., (1998) Biochim. Biophys. Acta 1414, 127-139; and peptides referred to as SKP or SN50 (Lin et al. (1995) J. Biol. Chem. 270, 14255-14258) or SP1068 (Rojas et al. (1996) J. Biol. Chem. 271, 27456-27461).

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “treatment” or “treating” encompasses prophylaxis and/or therapy. Accordingly the compositions and methods of the present invention are not limited to therapeutic applications and can be used in prophylaxis ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition associated with ocular angiogenesis, including alleviating symptoms of such diseases.

As used herein, the term “wild-type” refers to the genotype and phenotype that is characteristic of most of the members of a species occurring naturally and contrasting with the genotype and phenotype of a mutant.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates to compositions and methods for inhibiting ocular angiogenesis in a subject. Ocular angiogenesis is the formation of blood vessels in the eye, which can lead to visual impairment associated with a variety of ophthalmic disorders. The methods of the invention comprise administering to the subject an agent comprising a caveolin scaffolding domain. In one embodiment, the methods comprise administering an agent comprising a fusion peptide, wherein the fusion peptide comprises a caveolin scaffolding domain and a membrane translocation domain. In one embodiment, the membrane translocation domain is the antennapedia internalization sequence (RQIKIWFQNRRMKWKK) (SEQ ID NO: 12) from the antennapedia homeodomain. Agents useful for the methods of the present invention can be found in U.S. Pat. No. 7,494,976, the content of which is incorporated herein by reference. In some instances the agent comprising the antennapedia internalization sequence and a caveolin scaffolding domain is referred herein as AP-Cav or Cavtratin. In one embodiment, the AP-Cav or Cavtratin fusion peptide comprises SEQ ID NO: 12 and at least one amino acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, and SEQ ID NO: 11. In one embodiment AP-Cav or Cavtratin peptide has an amino acid sequence of SEQ ID NO: 13.

In one embodiment, the polypeptide of the invention can be prepared using standard solid phase (or solution phase) peptide synthesis methods, as is known in the art. In addition, the DNA encoding these polypeptides may be synthesized using commercially available oligonucleotide synthesis instrumentation and produced recombinantly using standard recombinant production systems. The production using solid phase peptide synthesis is necessitated if non-gene-encoded amino acids are to be included.

The present invention further provides using the isolated nucleic acid molecules that encode the polypeptide having the fusion peptides and conservative nucleotide substitutions thereof, preferably in isolated form to generate the compositions of the invention. Conservative nucleotide substitutions include nucleotide substitutions which do not affect the coding for a particular amino acid as most amino acids have more than one codon. Conservative nucleotide substitutions therefore also include silent mutations and differential codon usage.

In one embodiment, methods of the invention can be used to inhibit, to prevent and to treat a number of diseases and disorders marked by the development of ophthalmic neovascularization and related disorders. According to the present invention, the ophthalmic neovascularization and related disorders thereof (or disease or condition) are for example macular edema, ischemic retinopathy, intraocular neovascularization, age-related macular degeneration (AMD) and more specifically exudative AMD, corneal neovascularization, retinal neovascularization, choroidal neovascularization, retinopathy of prematurity, traumatic eye injury, diabetic macular edema, diabetic retina ischemia, diabetic retinal oedema, proliferative diabetic retinopathy, birdshot disease, multifocal choroiditis and any neovascularization associated with any pathological condition of the eye.

### Methods of Treatment

It has been demonstrated herein that the intraocular administration of the Cavtratin fusion peptide has the unexpected effect of reducing ocular angiogenesis, vascular leakage, and edema. Further it is shown herein that co-administration of cavtratin and anti-VEGF has the unexpected synergistic effect of enhancing the reduction of ocular angiogenesis, compared to each compound added alone. Therefore, the present invention relates to methods of inhibiting ocular angiogenesis, vascular leakage, and edema in a subject. Further, the present invention includes methods for treating and preventing ophthalmic conditions with an ocular angiogenesis, vascular leakage, or edema component (e.g. wet Age-related macular degeneration (AMD)).

The present invention relates to methods for the treatment of ocular related conditions or diseases, such as age-related macular degeneration (AMD), diabetic retinopathy, ocular angiogenesis (such as ocular neovascularization affecting choroidal, corneal, or retinal tissue), and other ocular conditions involving complement activation. Treatment of AMD includes both the dry and wet forms of AMD.

As provided in the Examples, compositions comprising a caveolin scaffolding domain may be used to modulate biological and pathologic processes (e.g., those associated with ocular angiogenesis).

Pathological processes refer to a category of biological processes which produce a deleterious effect. As used herein, an agent is said to modulate a pathological process when the agent reduces the degree or severity of the process. For instance, ocular angiogenic activity may be prevented or pathological processes modulated by the administration of agents which reduce, promote or modulate in some way the processes mediating ocular angiogenesis. The methods of the invention can therefore use an agent comprising a caveolin scaffolding domain to treat and prevent diseases with an ocular angiogenesis component. Such disease include but are not limited to; macular degeneration, diabetic retinopathy, diabetic macular edema, diabetic retina ischemia, diabetic retina oedema, proliferative diabetic retinopathy, birdshot disease, multifocal choroiditis, corneal graft rejection, corneal neovascularization, retinopathy of premature infants, retinal vein occlusion, neovascular glaucoma and sickle cell retinopathy, ischemic retinopathy, macular edema, intraocular neovascularization, choroidal neovascularization, pseudoxanthoma elasticum, optic disc drusen, traumatic eye injury and any other pathological condition of the eye.

Age-related macular degeneration (AMD) is a medical condition usually of older adults resulting in loss of vision in the center of the visual field (the macula) because of damage to the retina. AMD occurs in “dry” and “wet” forms, and is a major cause of blindness in the elderly (>50 years). In the advanced stages, AMD can make it difficult or impossible to read or recognize faces, although enough peripheral vision remains unaffected to allow other activities of daily life. The inner layer of the eye is the retina, which contains nerves that communicate sight. Behind the retina is the choroid, which contains the blood supply to the retina. There are two forms of AMD: dry and wet. The “dry” form of AMD (most common), results from atrophy of the retinal pigment epithelial layer below the retina, which causes vision loss through loss of photoreceptors (rods and cones) in the central part of the eye. In the dry form, cellular debris called “drusen” accumulate between the retina and the choroid, leading to retinal degeneration. While no treatment is available for this condition, vitamin supplements with high doses of antioxidants, lutein and zeaxanthin, have been suggested by the National Eye Institute and others to slow the progression of dry macular degeneration and, in some patients, improve visual acuity. As such, there is a need for new therapies to treat “dry” AMD.

In the wet (exudative) form, which is more severe, blood vessels grow outward from the choroid behind the retina, leading to retinal degeneration. Specifically, neovascular or exudative AMD, the “wet” form of advanced AMD, causes vision loss due to abnormal blood vessel growth (choroidal neovascularization) in the choriocapillaris, through Bruch's membrane, ultimately leading to blood and protein leakage below the macula. Wet AMD is typically treated with laser coagulation, photodynamic therapy, and with medications that stop and sometimes reverse the growth of blood vessels, or in combination. Bleeding, leaking, and scarring from the membrane and its blood vessels eventually cause irreversible damage to the photoreceptors and rapid vision loss if left untreated. Until recently, no effective treatments were known for wet macular degeneration. However, new drugs, called anti-angiogenics or anti-VEGF (anti-Vascular Endothelial Growth Factor) agents, can cause regression of the abnormal blood vessels and improvement of vision when injected directly into the vitreous humor of the eye. The injections can be painful and frequently have to be repeated on a monthly or bi-monthly basis. Examples of these agents include ranibizumab (trade name Lucentis®), bevacizumab (trade name Avastin®, a close chemical relative of ranibizumab) and pegaptanib (trade name Macugen®). Photodynamic therapy has also been used to treat wet AMD. While therapies for “wet” AMD are available, there is a need for better therapies such as therapies requiring fewer intraocular injections and adjuvant therapies that would inhibit retinal neovascularization using pathways other than the VEGF pathway.

The methods of the present invention can comprise administering agents comprising a caveolin scaffolding domain alone, or in combination with other agents that modulate a particular pathological process. For example, agents comprising a caveolin scaffolding domain can be administered in combination with one or more anti-inflammatory agents. As used herein, two agents are said to be administered in combination when the two agents are administered simultaneously or are administered independently in a fashion such that the agents will act at the same time. For example, in one embodiment, the methods of the invention comprise administering an agent comprising a caveolin scaffolding domain in combination with compounds capable of inhibiting the activity of vascular endothelial growth factor (VEGF). In one embodiment, the actions of an agent comprising a caveolin scaffolding domain are synergistic with the actions of compounds that inhibit VEGF. Such compounds include compounds capable of binding VEGF, including small organic molecules, antibodies or antibody fragments specific to VEGF, peptides, cyclic peptides, nucleic acids, antisense nucleic acids, RNAi, and ribozymes that inhibit VEGF expression at the nucleic acid level. Examples of compounds that inhibits VEGF are nucleic acid ligands of VEGF, such as those described in U.S. Pat. No. 6,168,778 or U.S. Pat. No. 6,147,204, EYE001 (previously referred to as NX1838) which is a modified, pegylated aptamer that binds with high affinity to the major soluble human VEGF isoform; VEGF polypeptides (e.g. U.S. Pat. No. 6,270,933 and WO 99/47677); oligonucleotides that inhibit VEGF expression at the nucleic acid level, for example antisense RNAs (e.g. U.S. Pat. No. 5,710,136; U.S. Pat. No. 5,661,135; U.S. Pat. No. 5,641,756; U.S. Pat. No. 5,639,872; and U.S. Pat. No. 5,639,736). Other examples of inhibitors of VEGF signaling known in the art (see introduction of the present invention) include, e.g., ZD6474 (Tuccillo et al., 2005, Olin Cancer Res., 11, 1268-76); COX-2, Tie2 receptor, angiopoietin, and neuropilin inhibitors; pigment epithelium-derived factor (PEDF), endostatin, and angiostatin, soluble fins-like tyrosine kinase 1 (sFltl) polypeptides or polynucleotides (Harris et al., 2001, Olin Cancer Res., 7, 1992-1997; U.S. Pat. No. 5,861,484); PTK787/ZK222 584; KRN633 (Maier et al., 2004, Mol Cancer Ther., 3, 1639-1649); VEGF-Trap® (Regeneron); and Alpha2-antiplasmin (Matsuno et al, 2003, Blood, 120, 3621-3628). For reviews of VEGF and its inhibitors, see, e.g., Campochiaro, 2004, Expert Opin Biol Ther., 4, 1395-1402; Ferrara, 2004, Endocr. Rev., 25, 581-611; the content of which are incorporated herein by reference). According to preferred embodiment, compounds that inhibit VEGF are antibodies to, or antibody fragments thereof, or aptamers of VEGF or a related family member. Preferred examples are anti-VEGF antibodies, e.g. Avastin™ (also reviewed as bevacizumab, Genentech), or fragments thereof, e.g. Lucentis™ (also reviewed as rhuFAb V2 or AMD-Fab; ranibizumab, Genentech), and other anti-VEGF compounds such as VEGF inhibitory aptamers, e.g., Macugen™ (also reviewed as pegaptanib sodium, anti-VEGF aptamer or EYE001, Pfizer). According to one embodiment, the compound that inhibits VEGF can further be an immunosuppressant compound, and may be selected from the group consisting of calcineurin inhibitors and mTOR inhibitors.

The methods of the invention are also readily adaptable for use in assay systems, e.g., assaying ocular angiogenesis and properties thereof.

The disorders treatable by means of the present invention occur in mammals. Mammals include, for example, humans, as well as pet animals such as dogs and cats, laboratory animals such as rats and mice, and farm animals such as horses and cows.

In one embodiment, the compositions of the invention can be administered directly to the eye, such as topically, subconjunctivally, or intravitreally, or they may be administered at a site distal to the eye, such as intravenously or subcutaneously.

In one embodiment, the methods of the present invention can comprise the delivery of agents comprising a caveolin scaffolding domain administered via transocular, intraocular, transnasal, intranasal, parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

### Formulations

The composition of the invention can be administered as a mixture, as an admixture, in the same ophthalmic composition, in separate formulations, in extended release formulations, liposomes, microcapsules, or any of the previously described embodiments.

The composition of the invention can also be administered as a slow release formulation, with a carrier formulation such as microspheres, microcapsules, liposomes, etc., as a topical ointment or solution, an intravenous solution or suspension, or in an intraocular injection, as known to one skilled in the art to treat or prevent ophthalmic disorders. By “slow release”, “time-release”, “sustained release” or “controlled release” is meant that the therapeutically active component is released from the formulation at a controlled rate such that therapeutically beneficial levels (but below toxic levels) of the component are maintained over an extended period of time ranging from e.g., about 12 to about 24 hours, thus, providing, for example, a 12 hour or a 24 hour dosage form. A time-release drug delivery system may be administered intraocularly to result in sustained release of the combination product over a period of time. The combination product may be in the form of a vehicle, such as a micro- or macro-capsule or matrix of biocompatible polymers such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SATE), and other polymers such as those disclosed in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety, or lipids that may be formulated as microspheres or liposomes. A microscopic or macroscopic ophthalmic composition may be administered through a needle, or may be implanted by suturing within the eye, e.g., intravitreal cavity or sub-retinal space. Delayed or extended release properties may be provided through various formulations of the vehicle (coated or uncoated microsphere, coated or uncoated capsule, lipid or polymer components, unilamellar or multilamellar structure, and combinations of the above, etc.). The formulation and loading of microspheres, microcapsules, liposomes, etc. and their ocular implantation are standard techniques known by one skilled in the art.

The invention also provides a method for the treatment or prophylaxis of ophthalmic disorders related to neovascularization. In one embodiment, the method comprises the step of administering a composition of the invention in a biocompatible, biodegradable matrix, for example in the form of a gel or polymer which is preferably suited for insertion into the retina or into a cavity of the eye, anterior or posterior, as an implant. In the case that the combination product is delivered as an implant, it may be incorporated in any known biocompatible biodegradable matrix as a liquid, or in the form, for example, of a micelle using known chemistry or as microparticles.

### Pharmaceutical Preparations

The invention provides a pharmaceutical that is effective for ocular disorders associated with excess angiogenesis, i.e., ocular neovascular diseases. Examples of the ocular neovascular diseases may include ocular angiogenic disorders such as (wet) age-related macular degeneration (wet-AMID), branch retinal vein occlusion (BRVO), central retinal vein occlusion (CRVO), diabetic retinopathy, neovascular glaucoma, myopic choroidal neovascularization, retinitis pigmentosa, retinopathy of prematurity and photocoagulation-induced choroidal neovascularization.

In one embodiment, the invention provides a pharmaceutical that is effective for disorders associated with increased ocular vascular permeability. Examples of the disorders accompanied by increased ocular vascular permeability may include wet age-related macular degeneration (wet-AMID), branch retinal vein occlusion (BRVO), central retinal vein occlusion (CRVO), diabetic maculopathy, retinitis pigmentosa, diabetic retinopathy and edema due to retinal photocoagulation (panretinal photocoagulation, grid photocoagulation, Grid Pattern photocoagulation). The ocular angiogenic disorders and disorders accompanied by increased ocular vascular permeability are not limited to these disorders.

The invention also includes administration of pharmaceutical compositions comprising the agents comprising a caveolin scaffolding domain together with a pharmaceutically acceptable carrier. Pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical carriers are described in Gennaro et al., (1995) Remington's Pharmaceutical Sciences, Mack Publishing Company. In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil or synthetic fatty acid esters, for example, ethyl oleate or triglycerides. Aqueous injection suspensions may contain substances which increase the viscosity of the suspension include, for example, sodium carboxymethyl cellulose, sorbitol, and dextran. Optionally, the suspension may also contain stabilizers.

Liposomes can also be used to encapsulate a drug for delivery into the cell, in particular, hydrophobic drugs. In one embodiment, the invention provides pharmaceutical compositions comprising a peptide-liposome complex in a pharmaceutically compatible vehicle or carrier. Liposomes are spherical colloidal structures in which an internal aqueous phase is surrounded by one or more phospholipid bilayers. The use of liposomes as drug delivery systems has been disclosed in U.S. Pat. Nos. 3,993,754; 4,235,871; 4,356,167). The compositions are formulated for, preferably, intravenous administration to a human patient in need of the effective delivery of the agent. These complexes are appropriately sized so that they are distributed throughout the body following intravenous administration. In addition, these liposomes can be modified such that they are uniquely suited for the delivery of a specific drug as described in U.S. Pat. No. 5,785,976.

In another embodiment, the invention relates to therapeutic methods comprising the administration to a subject of a therapeutically effective amount of a pharmaceutical composition comprising a peptide in a pharmaceutically acceptable vehicle. As set forth in detail herein, treatment of ocular angiogenesis via the intraocular administration of an agent comprising the Cavtratin peptide is an important embodiment of this aspect of the invention. In one embodiment, the agent comprising a caveolin scaffolding domain is diluted into acetic acid, dimethyl sulfoxide (DMSO), carboxy methylcellulose (CMC), or other excipient. In one embodiment, the agent is administered via intraocular injection.

In another embodiment, micelles can be prepared by a wide variety of methods using an equally diverse group of compounds for effective delivery of the peptides of the invention. Many forms of polymers have been used to form micelles for effective delivery of peptides. These polymers have generally been block copolymers. Such micelle forming polymeric formulations have generally been designed to behave in a pH independent manner and have comprised nonionic polymers or copolymers (Ropert et al., (1992) Biochem. Biophys. Res. Commun. 187, 379-885; Seki et al., (1984) Macromolecules 17, 1692-1698; Kwon & Kazunori, (1995) Adv. Drug Deliv. Rev. 16, 295-309). In one embodiment, the invention includes methods for the effective delivery of caveolin peptides by providing a formulation containing micelles that include a caveolin peptide which is released from the micelle following administration.

The pharmaceutical formulations for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

The methods of the present invention include delivery of agents comprising a caveolin scaffolding domain which are administered via transocular, intraocular, transnasal, intranasal, parenteral, subcutaneous, intravenous, intramuscular, intraperitoneal, transdermal or buccal routes. Alternatively, or concurrently, administration may be by the oral route or by inhalation or lavage, directly to the lungs. The dosage administered will be dependent upon the age, health, and weight of the recipient, kind of concurrent treatment, if any, frequency of treatment, and the nature of the effect desired.

The methods of the present invention include delivery of agents comprising a caveolin scaffolding domain which are administered systemically or topically, depending on such considerations as the condition to be treated, need for site-specific treatment, quantity of drug to be administered and similar considerations.

Topical administration may be used. Any common topical formation such as a solution, suspension, gel, ointment or salve and the like may be employed. Preparation of such topical formulations are well described in the art of pharmaceutical formulations as exemplified, for example, by Remington's Pharmaceutical Sciences. For topical application, these compounds could also be administered as a powder or spray, particularly in aerosol form. The active ingredient may be administered in pharmaceutical compositions adapted for systemic administration. As is known, if a drug is to be administered systemically, it may be confected as a powder, pill, tablet or the like or as a syrup or elixir for oral administration. For intravenous, intraperitoneal or intra-lesional administration, the compound will be prepared as a solution or suspension capable of being administered by injection. In certain cases, it may be useful to formulate these compounds in suppository form or as an extended release formulation for deposit under the skin or intramuscular injection. In a preferred embodiment, the compounds of this invention may be administered by inhalation. For inhalation therapy the compound may be in a solution useful for administration by metered dose inhalers or in a form suitable for a dry powder inhaler.

An effective amount is that amount which will modulate the activity or alter the level of a target protein. A given effective amount will vary from condition to condition and in certain instances may vary with the severity of the condition being treated and the patient's susceptibility to treatment. Accordingly, a given effective amount will be best determined at the time and place through routine experimentation.

In one embodiment, the methods of the present invention comprise administration of agents comprising a caveolin scaffolding domain by intraocular injection, although other modes of administration may be effective. Typically, the methods comprise delivery of the agent intraocularly (by chemical delivery system or invasive device) to an individual. However, the invention is not limited to intraocular delivery in that it also includes topically (extraocular application) or systemically (e.g. oral or other parenteral route such as for example subcutaneous administration). Parenteral administration is used in appropriate circumstances apparent to the practitioner. Preferably, the agents are administered in unit dosage forms suitable for single administration of precise dosage amounts.

Delivery to areas within the eye, in situ can be accomplished by injection, cannula or other invasive device designed to introduce precisely metered amounts of a desired ophthalmic composition to a particular compartment or tissue within the eye (e.g. posterior chamber or retina). An intraocular injection may be into the vitreous (intravitreal), or under the conjunctiva (subconjunctival), or behind the eye (retrobulbar), into the sclera, or under the Capsule of Tenon (sub-Tenon), and may be in a depot form. Other intraocular routes of administration and injection sites and forms are also contemplated and are within the scope of the invention. In one embodiment the methods comprise delivery of agents by sub-retinal injection.

In one embodiment, the agents are intraocularly injected (e.g., into the vitreous or sub retinal) to treat or prevent an ophthalmic condition. When administering the agents by intraocular injection, the active agents should be concentrated to minimize the volume for injection. Volumes such as this may require compensatory drainage of the vitreous fluid to prevent increases in intraocular pressure and leakage of the injected fluid through the opening formed by the delivery needle

Intraocular injection may be achieved by a variety of methods well known in the art. For example, the eye may be washed with a sterilizing agent such as Betadine® and the agents are injected in an appropriate carrier with a fine gauge needle (e.g. 27 gauge) at a position in the eye such that the compound will settle to the posterior pole towards the ventral surface. It may be necessary to prepare the eye for injection by application of positive pressure prior to injection. In some cases, preliminary vitrectomy may be necessary. Local anaesthetic or general anaesthetic may be necessary.

The syringe used in practicing the method of this invention is suitably one which can accommodate a 21 to 40 gauge needle and is preferably of a small volume, for example 1.5 ml, or more preferably 0.1 ml. Although it is possible that the needle and syringe may be of the type where the needle is removable from the syringe, it is preferred that the arrangement is of a unitary syringe/needle construction. This would clearly limit the possibility of disengagement of the needle from the syringe. It is also preferred that the arrangement be tamper evident. The agents to be administered by way of the methods of the invention may therefore be provided in the form of a single unit dose, or separated unit doses each containing part of the combination product, in a pre-prepared syringe ready for administration.

A suitable style of syringe is, for example, sold under the name of Uniject® manufactured by Becton Dickinson and Company. In this style of syringe, the material is expelled through the needle into the eye by pressure applied to the sides of a pliable reservoir supplying the needle, rather than by a plunger. As the name implies, the construction of the reservoir and needle forms a single unit.

Topical application of the agent comprising a caveolin scaffolding domain for the treatment or prevention of ophthalmic disorders may be as ointment, gel or eye drops. The topical ophthalmic composition may further be an in situ gellable aqueous formulation. Such a formulation comprises a gelling agent in a concentration effective to promote gelling upon contact with the eye or with lacrimal fluid in the exterior of the eye. Suitable gelling agents include, but are not limited to, thermosetting polymers such as tetra-substituted ethylene diamine block copolymers of ethylene oxide and propylene oxide (e.g., poloxamine); polycarbophil; and polysaccharides such as gellan, carrageenan (e.g., kappa-carrageenan and iota-carrageenan), chitosan and alginate gums.

The phrase “in situ gellable” as used herein embraces not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye.

To prepare a topical ophthalmic composition for the treatment of ophthalmic disorders, a therapeutically effective amount of an agent comprising a caveolin scaffolding domain is placed in an opthalmological vehicle as is known in the art. For example, topical ophthalmic formulations containing steroids are disclosed in U.S. Pat. No. 5,041,434, whilst sustained release ophthalmic formulations of an ophthalmic drug and a high molecular weight polymer to form a highly viscous gel have been described in U.S. Pat. No. 4,271,143 and U.S. Pat. No. 4,407,792. Further GB 2007091 describes an ophthalmic composition in the form of a gel comprising an aqueous solution of a carboxyvinyl polymer, a water-soluble basic substance and an ophthalmic drug. Alternatively, U.S. Pat. No. 4,615,697, discloses a controlled release composition and method of use based on a bioadhesive and a treating agent, such as an anti-inflammatory agent.

The amount of the agent to be administered and the concentration of the agent in the topical ophthalmic combination product used in the method depend upon the diluent, delivery system or device, the clinical condition of the patient, the side effects and the stability of the compound in the formulation. Thus, the physician employs the appropriate preparation containing the appropriate concentration of the agent and selects the amount of formulation administered, depending upon clinical experience with the patient in question or with similar patients.

In one embodiment, the agent comprising a caveolin scaffolding domain may be administered along with at least one other agent (e.g. a composition capable of inhibiting the activity of VEGF). The active agents may be administered as a mixture, as an admixture, in the same ophthalmic composition, in separate formulations, in extended release formulations, liposomes, microcapsules, or any of the previously described embodiments.

The agent comprising a caveolin scaffolding domain may be also administered as a slow release formulation, with a carrier formulation such as microspheres, microcapsules, liposomes, etc., as a topical ointment or solution, an intravenous solution or suspension, or in an intraocular injection, as known to one skilled in the art to treat or prevent ophthalmic disorders. By “slow release”, “time-release”, “sustained release” or “controlled release” is meant that the therapeutically active component is released from the formulation at a controlled rate such that therapeutically beneficial levels (but below toxic levels) of the component are maintained over an extended period of time ranging from e.g., about 12 to about 24 hours, thus, providing, for example, a 12 hour or a 24 hour dosage form. A time-release drug delivery system may be administered intraocularly to result in sustained release of the agent over a period of time. The agent may be in the form of a vehicle, such as a micro- or macro-capsule or matrix of biocompatible polymers such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SATE), and other polymers such as those disclosed in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety, or lipids that may be formulated as microspheres or liposomes. A microscopic or macroscopic ophthalmic composition may be administered through a needle, or may be implanted by suturing within the eye, e.g. intravitreal cavity or sub-retinal space. Delayed or extended release properties may be provided through various formulations of the vehicle (coated or uncoated microsphere, coated or uncoated capsule, lipid or polymer components, unilamellar or multilamellar structure, and combinations of the above, etc.). The formulation and loading of microspheres, microcapsules, liposomes, etc. and their ocular implantation are standard techniques known by one skilled in the art.

The invention also provides a method for the treatment or prophylaxis of ophthalmic disorders related to neovascularization, said method comprising the step of administering an agent comprising a caveolin scaffolding domain within in a biocompatible, biodegradable matrix, for example in the form of a gel or polymer which is preferably suited for insertion into the retina or into a cavity of the eye, anterior or posterior, as an implant. In the case that the agent is delivered as an implant, it may be incorporated in any known biocompatible biodegradable matrix as a liquid, or in the form, for example, of a micelle using known chemistry or as microparticles.

Slow or extended-release delivery systems include any of a number of biopolymers (biological-based systems), systems employing liposomes, colloids, resins, and other polymeric delivery systems or compartmentalized reservoirs, can be utilized with the compositions described herein to provide a continuous or long term source of therapeutic compound.

In one form, implants used in the method of the present invention are formulated with an agent comprising a caveolin scaffolding domain entrapped within the bio-erodible polymer matrix. Release of the agent is achieved by erosion of the polymer followed by exposure of previously entrapped compound to the vitreous, and subsequent dissolution and release of agent. The release kinetics achieved by this form of drug release are different than that achieved through formulations which release drug through polymer swelling, such as with hydrogels such as methylcellulose. In that case, the active agent is not released through polymer erosion, but through polymer swelling, which releases active compound as liquid diffuses through the pathways exposed. The parameters which determine the release kinetics include the size of the active agent particles, the water solubility of the active compound, the ratio of active agent to polymer, the method of manufacture, the surface area exposed, and the erosion rate of the polymer.

Exemplary biocompatible, non-biodegradable polymers of particular interest include polycarbamates or polyureas, particularly polyurethanes, polymers which may be cross-linked to produce non-biodegradable polymers such as cross-linked polyvinyl acetate) and the like. Also of particular interest are ethylene-vinyl ester copolymers having an ester content of 4% to 80% such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer.

Additional exemplary naturally occurring or synthetic non-biodegradable polymeric materials include poly(methylmethacrylate), poly(butylmethacrylate), plasticized poly(vinylchloride), plasticized poly(amides), plasticized nylon, plasticized soft nylon, plasticized poly(ethylene terephthalate), natural rubber, silicone, poly(isoprene), poly(isobutylene), poly(butadiene), poly(ethylene), poly(tetrafluoroethylene), poly(vinylidene chloride), polyacrylonitrile, cross-linked poly(vinylpyrrolidone), poly(trifluorochloroethylene), chlorinated poly(ethylene), poly(4,4′-isopropylidene diphenylene carbonate), vinylidene chloride-acrylonitrile copolymer, vinyl chloridediethyl fumarate copolymer, silicone, silicone rubbers (especially the medical grade), poly(dimethylsiloxanes), ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(vinyl-olefins), poly(styrene), poly(halo-olefins), poly(vinyls), poly(acrylate), poly(methacrylate), poly(oxides), poly(esters), poly(amides), and poly(carbonates).

Diffusion of the agent comprising a caveolin scaffolding domain from the implant may also be controlled by the structure of the implant. For example, diffusion of the agent from the implant may be controlled by means of a membrane affixed to the polymer layer comprising the drug. The membrane layer will be positioned intermediate to the polymer layer comprising the agent and the desired site of therapy.

The skilled reader will appreciate that the duration over which any of the ophthalmic combination product used in the method of the invention will dwell in the ocular environment will depend, inter alia, on such factors as the physicochemical and/or pharmacological properties of the compounds employed in the formulation, the concentration of the compound employed, the bioavailability of the compound, the disease to be treated, the mode of administration and the preferred longevity of the treatment. Where that balance is struck will often depend on the longevity of the effect required in the eye and the ailment being treated.

The frequency of treatment according to the method of the invention is determined according to the disease being treated, the deliverable concentration of the agent and the method of delivery. If delivering the combination product by intravitreal injection, the dosage frequency may be monthly. Preferably, the dosage frequency is every three months. The frequency of dosage may also be determined by observation, with the dosage being delivered when the previously delivered combination product is visibly cleared. In general, an effective amount of the compound is that which provides either subjective relief of symptoms or an objectively identifiable improvement as noted by the clinician or other qualified observer.

The administered agent, delivered in the method of the present invention to prevent or treat ophthalmic disorders will preferably have dwell times from hours to many months and possibly years, although the latter time period requires special delivery systems to attain such duration and/or alternatively requires repetitive administrations. Most preferably the agent for use in the method of the invention will have a dwell time (i.e. duration in the eye) of hours (i.e. 1 to 24 hours), days (i.e. 1, 2, 3, 4, 5, 6 or 7 days) or weeks (i.e. 1, 2, 3, 4 weeks). Alternatively, the agent will have a dwell time of at least a few months such as, 1 month, 2 months, 3 months, with dwell times of greater than 4, 5, 6, 7 to 12 months being achievable.

If desired, the method or use of the invention can be carried out alone, or in conjunction with one or more conventional therapeutic modalities (such as photodynamic therapy, laser surgery, laser photocoagulation or one or more biological or pharmaceutical treatments. These methods are well known from the skilled man in the art and widely disclosed in the literature). The use of multiple therapeutic approaches provides the patient with a broader based intervention. In one embodiment, the method of the invention can be preceded or followed by a surgical intervention. In another embodiment, it can be preceded or followed by photodynamic therapy, laser surgery, and laser photocoagulation. Those skilled in the art can readily formulate appropriate therapy protocols and parameters which can be used.

In practicing the methods of this invention, the agents comprising a caveolin scaffolding domain may be used alone or in combination, or in combination with other therapeutic or diagnostic agents. In certain preferred embodiments, the agents comprising a caveolin scaffolding domain may be co-administered along with other compounds typically prescribed for these conditions according to generally accepted medical practice. The compounds of this invention can be utilized in vivo, ordinarily in mammals, preferably in humans.

In still another embodiment, the agents comprising a caveolin scaffolding domain may be coupled to chemical moieties, including proteins that alter the functions or regulation of target proteins for therapeutic benefit. These proteins may include in combination other inhibitors of cytokines and growth factors that may offer additional therapeutic. In addition, the agents comprising a caveolin scaffolding domain may also be conjugated through phosphorylation to biotinylate, thioate, acetylate, iodinate using any of the cross-linking reagents well known in the art.

The agents comprising a caveolin scaffolding domain can also be coupled to molecules which enhance the transmembrane transport of peptide across a membrane having biotin or folate receptors that initiate transmembrane transport of receptor bound species. The method takes advantage of (1) the location and multiplicity of biotin and folate receptors on the membrane surfaces of most cells and (2) the associated receptor mediated transmembrane processes. Performance of the method involves formation of a complex between a ligand selected from biotin or other biotin receptor-binding compounds, and/or folic acid or other folate receptor-binding compounds, and the peptide. A cell membrane bearing biotin or folate receptors is contacted with this complex, thereby initiating receptor mediated transmembrane transport of the complex. The complex is allowed to contact the membrane surface bearing the corresponding receptors for a time sufficient to initiate and permit transmembrane transport of the complex. The transmembrane transport of the peptides can be promoted in plant, mammalian, and bacterial cells.

In one embodiment of this invention, the target receptor for the method of the present invention is the biotin receptor. Biotin is a necessary cellular nutrient that has been found to be preferentially bound by biotin receptor proteins associated with cellular membranes. Commercially available reagents are used to form a covalent complex between biotin and polynucleotides, proteins, or other desired exogenous molecules. According to one preferred embodiment of the present invention, a biotinylated peptide is brought into contact with a membrane having associated biotin receptors for a time sufficient to allow binding of the biotin moiety of the complex to a corresponding biotin receptor in the membrane. This binding triggers the initiation of cellular processes that results in transmembrane transport of the complex.

In an alternate but equally preferred embodiment of this invention, folate receptors are targeted to enhance cellular uptake of exogenous molecules. Folate binding receptors are found in most types of cells, and they have been demonstrated to bind and trigger cellular internalization of folates. Thus, folic acid and other art-recognized folate receptor-binding ligands can be chemically bonded to the peptides of the invention using art-recognized coupling techniques to provide a folate receptor-binding complex which is readily endocytosed into living cells. In accordance with this embodiment of the present invention, a folate-peptide complex is brought into contact with a membrane having associated folate receptors for a time sufficient to allow binding of the folate moiety of the complex to a corresponding folate receptor. Folate receptor-binding triggers the initiation of cellular processes that result in transmembrane transport of the complex.

The use of biotin and folate receptors is particularly useful for increasing the internalization efficiency (cellular uptake) of peptides that are normally resistant to cellular internalization. Peptides previously recognized as difficult to move across cell membranes can be internalized by a cell through the use of biotin and folate receptors. For example, blockade of eNOS with a caveolin peptide can be accomplished by coupling the caveolin peptide to either biotin or folates, and contacting the cells with the resulting complex for a time sufficient to promote cellular internalization. The use of biotin and folates complexes to enhance cellular uptake of complexed exogenous molecules has been demonstrated in vivo and in vitro (see U.S. Pat. No. 5,635,382).

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Cavtratin Reduces Ocular Angiogenesis

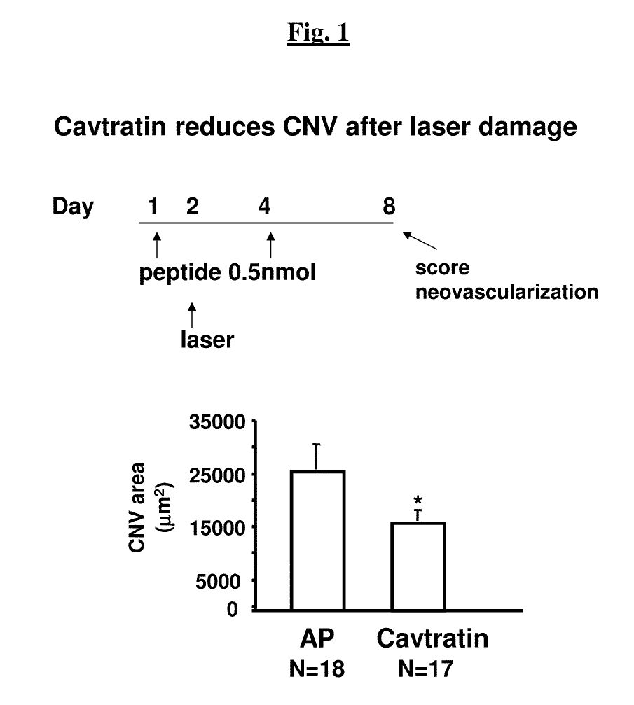

Experiments were designed to examine whether treatment with cavtratin is useful in disease models of excessive ocular angiogenesis. To induce choroidal neovascularization (CNV), a model for retinal angiogenesis in mice, three 532 nm diode laser spots were applied to each fundus of the eye using a coverslip as a contact lens. The lesions were placed between the retinal vessels 2-3 disc diameters from the optic nerve. Formation of a bubble indicates rupture of the Bruch's membrane, a critical factor in driving CNV. Cavtratin was dissolved into acetic acid and diluted into saline. As seen in FIG. 1, mice were intraocularly injected (1 microliter) with AP (control) or cavtratin (0.5 nmol) using saline as a vehicle, prior to laser damage then again 2 days after damage and mice sacrificed after 4 additional days. After treatment and injury, CNV was visualized by immunofluorescent labeling with isolectin b4, a marker for angiogenic blood vessels. CNV area was quantified as isolectin positive vessels per area measured. As seen in FIG. 1, cavtratin reduced CNV in this model (N=17-18 eyes, P<0.05).

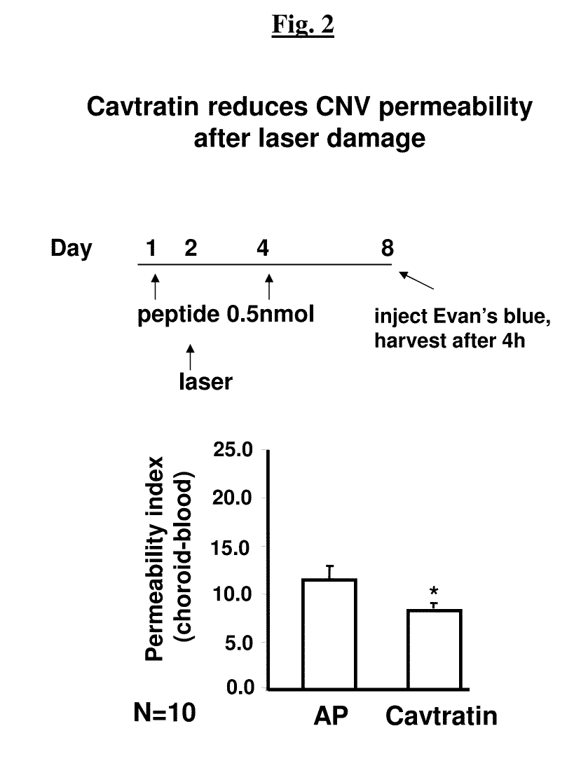

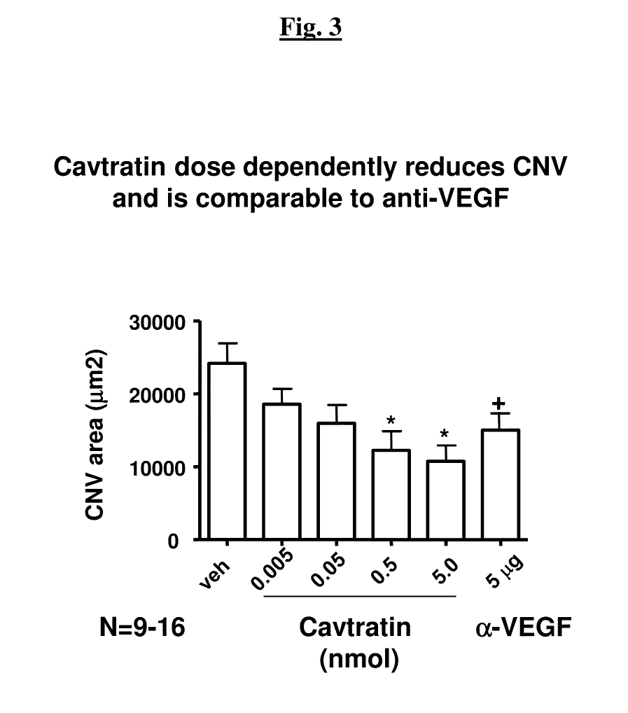

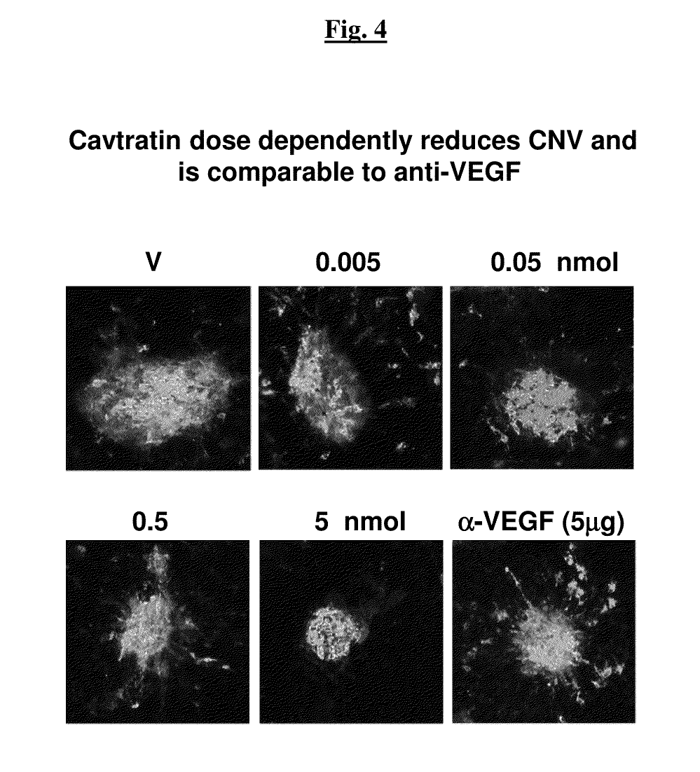

Since mechanistically cavtratin reduces vascular permeability, vascular leakage was examined after CNV damage. Using a similar protocol as above, cavtratin reduced vascular permeability as quantified by Evan's blue extravasation (FIG. 2). Further, as shown in FIG. 3 and FIG. 4, cavtratin dose-dependently reduces CNV using anti-VEGF administration as a comparator.

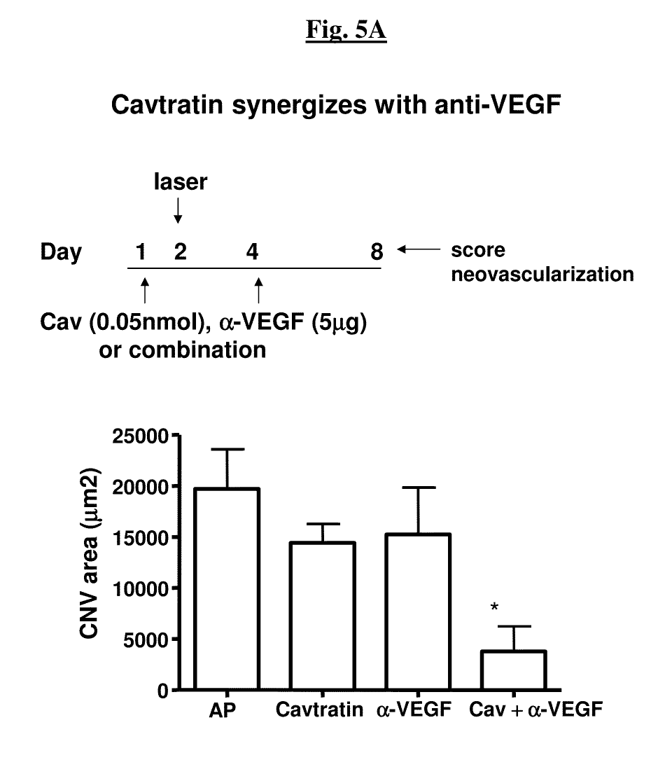

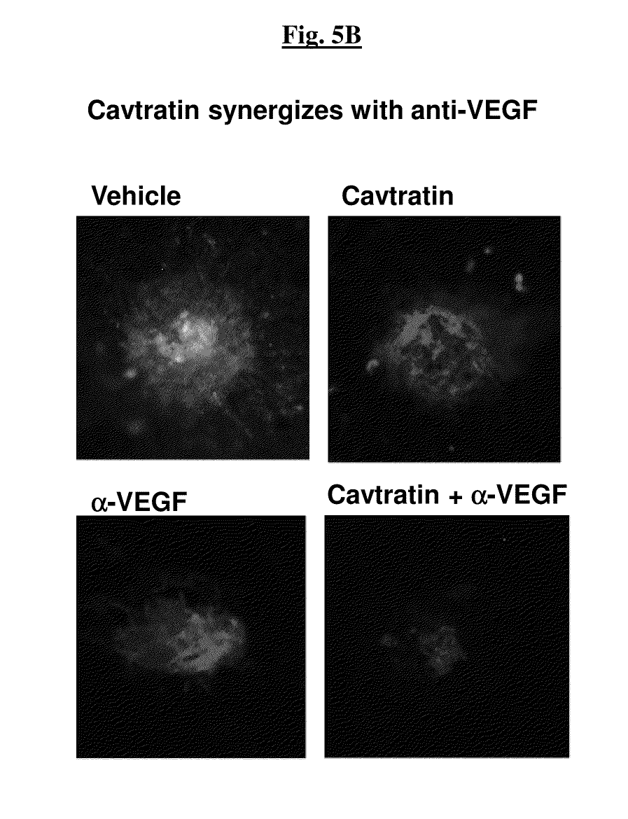

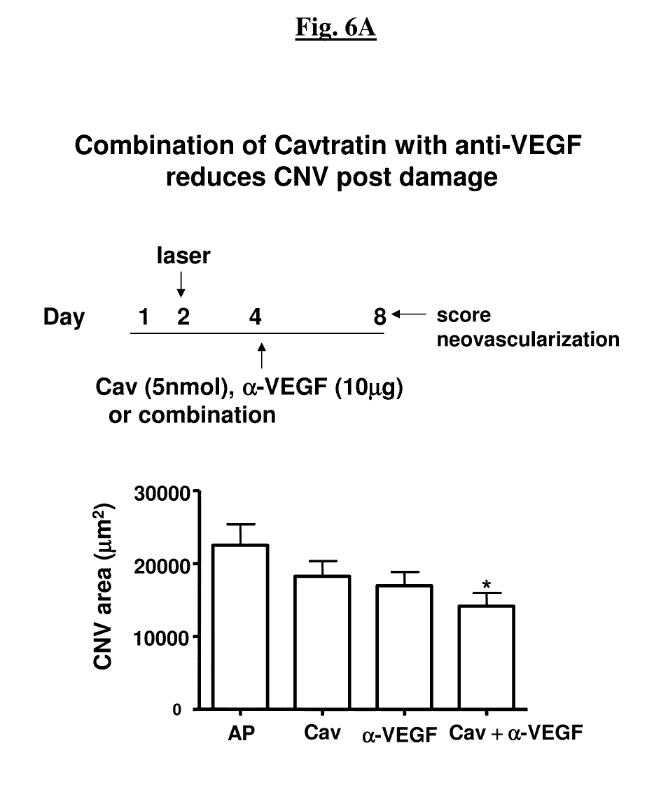

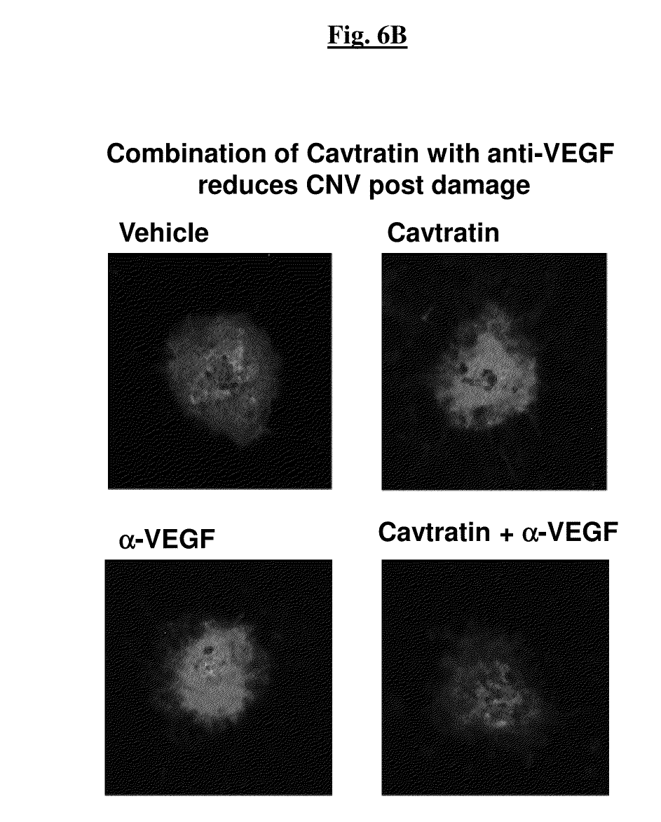

Further, it was examined whether the effects of cavtratin treatment synergizes with anti-VEGF. Mice were injected with either cavtratin (0.05 nmol) one day prior to laser damage, anti-VEGF (5 μg) 2 days after damage, or were treated with both cavtratin and anti-VEGF. As shown in FIGS. 5A-5B, when mice were sacrificed, 6 days post damage, and neovascularization was evaluated, the combination treatment of cavtratin and anti-VEGF significantly reduced CNV area compared to control, cavtratin alone, and anti-VEGF alone. Similarly, as shown in FIGS. 6A-6B, when 5 nmol cavtratin and 10 μg anti-VEGF were both administered 2 days post laser damage, the combination treatment significantly reduced CNV.

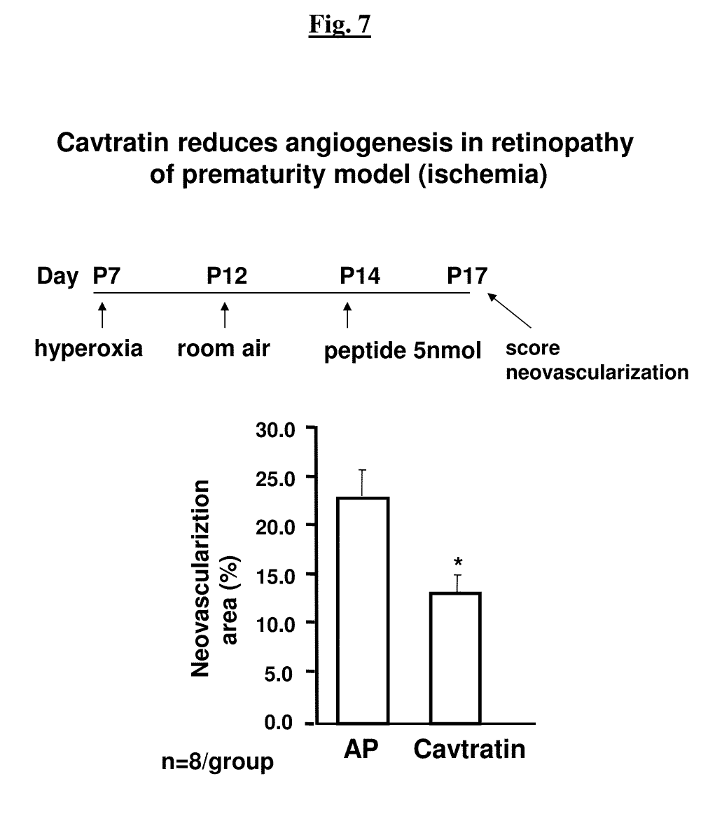

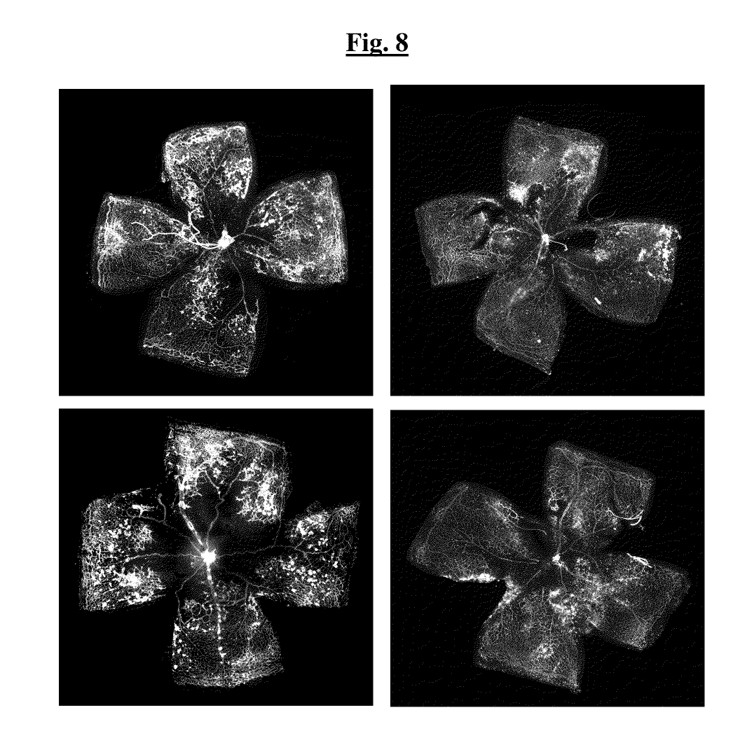

Next, cavtratin was tested in an additional model of ocular angiogenesis, retinopathy of prematurity model (ROP). Exposure to hyperoxia during early retinal development leads to the arrest or retardation of normal retinal vascular development. When the animals are returned to the normoxic environment, they are under a relative hypoxic situation where the retina now lacks its normal vasculature that is required to adequately support the neural tissue in normoxic conditions. This ischemic situation results in unregulated, abnormal neovascularization. The neovascular response is very consistent, reproducible and quantifiable, and it has become integral model for studying disease mechanisms and potential treatments for ischemic retinopathy. The mouse ROP model has become the most common model for studying pathologic angiogenesis and has been extended to the general study of ischemic retinopathies and related anti-angiogenic treatments. As seen in FIG. 7, cavtratin administration 2 days after switching from hyperoxia to room air results in an attenuation of extensive, ischemia triggered neovascualization. Cavtratin induced attenuation of ischemia triggered neovascularization is shown in FIG. 8, where the left panels are representative of two AP injected eyes and right panels are from cavtratin injections.

Overall, these results demonstrate that cavtratin is useful as a therapeutic for exaggerated ocular angiogenesis.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

1. A method of inhibiting ocular angiogenesis in a subject in need thereof, the method comprising administering to the subject an effective amount of an agent comprising at least one caveolin scaffolding domain selected from the group consisting of SEQ ID NOs: 1-11.

2. (canceled)

3. The method of claim 1, wherein the agent is a fusion peptide comprising at least one membrane translocation domain and the at least one caveolin scaffolding domain.

4. The method of claim 1, wherein the agent is administered via intraocular injection to the subject.

5. The method of claim 1, wherein the agent is administered in combination with an anti-angiogenic agent to the subject.

6. The method of claim 5, wherein the anti-angiogenic agent reduces the activity of vascular endothelial growth factor (VEGF).

7. A method of treating an ophthalmic condition in a subject in need thereof, the method comprising administering to the subject an effective amount of an agent comprising at least one caveolin scaffolding domain selected from the group consisting of SEQ ID NOs: 1-11, wherein the ophthalmic condition has at least one component selected from the group consisting of ocular angiogenesis, vascular leakage, and edema.

8. (canceled)

9. The method of claim 7, wherein the agent is a fusion peptide comprising at least one membrane translocation domain and the at least one caveolin scaffolding domain.

10. The method of claim 7, wherein an effective amount of the agent is administered via intraocular injection to the subject.

11. The method of claim 7, wherein the agent is administered in combination with an anti-angiogenic agent to the subject.

12. The method of claim 11, wherein the anti-angiogenic agent reduces the activity of VEGF.

13. The method of claim 7, wherein the ophthalmic condition is selected from the group consisting of macular degeneration, diabetic retinopathy, diabetic macular edema, diabetic retina ischemia, diabetic retina oedema, proliferative diabetic retinopathy, birdshot disease, multifocal choroiditis, corneal graft rejection, corneal neovascularization, retinopathy of premature infants, retinal vein occlusion, neovascular glaucoma and sickle cell retinopathy, ischemic retinopathy, macular edema, intraocular neovascularization, choroidal neovascularization, pseudoxanthoma elasticum, optic disc drusen, and traumatic eye injury.

14. A method of preventing an ophthalmic condition in a subject in need thereof, the method comprising administering to the subject an effective amount of an agent comprising a at least one caveolin scaffolding domain selected from the group consisting of SEQ ID NOs: 1-11, wherein the ophthalmic condition has at least one component selected from the group consisting of ocular angiogenesis, vascular leakage, and edema.

15. (canceled)

16. The method of claim 14, wherein the agent is a fusion peptide comprising at least one membrane translocation domain and the at least one caveolin scaffolding domain.

17. The method of claim 14, wherein an effective amount of the agent is administered via intraocular injection to the subject.

18. The method of claim 14, wherein the agent is administered in combination with an anti-angiogenic agent to the subject.

19. The method of claim 18, wherein the anti-angiogenic agent reduces the activity of VEGF.

20. The method of claim 14, wherein the ophthalmic condition is selected from the group consisting of macular degeneration, diabetic retinopathy, diabetic macular edema, diabetic retina ischemia, diabetic retina oedema, proliferative diabetic retinopathy, birdshot disease, multifocal choroiditis, corneal graft rejection, corneal neovascularization, retinopathy of premature infants, retinal vein occlusion, neovascular glaucoma and sickle cell retinopathy, ischemic retinopathy, macular edema, intraocular neovascularization, choroidal neovascularization, pseudoxanthoma elasticum, optic disc drusen, and traumatic eye injury.

21. A method of reducing vascular leakage and edema in a subject in need thereof, the method comprising administering to the subject an effective amount of an agent comprising a at least one caveolin scaffolding domain selected from the group consisting of SEQ ID NOs: 1-11.

22. (canceled)

23. The method of claim 21, wherein the agent is a fusion peptide comprising at least one membrane translocation domain and the at least one caveolin scaffolding domain.

24. The method of claim 21, wherein an effective amount of the agent is administered via intraocular injection to the subject.

25. The method of claim 21, wherein the agent is administered in combination with an anti-angiogenic agent to the subject.

26. The method of claim 25, wherein the anti-angiogenic agent reduces the activity of vascular endothelial growth factor (VEGF).
